(19) United States
(12) Patent Application Publication
(10) Pub. No.: US 2026/0102208 A1
(43) Pub. Date: Apr. 16, 2026

(54) SYSTEMS AND METHODS FOR PLANNING CORONARY INTERVENTIONS

(71) Applicant: KONINKLIJKE PHILIPS N.V., EINDHOVEN (NL)

(72) Inventors: MICHAEL GRASS, BUCHHOLZ IN DER NORDHEIDE (DE); CHRISTIAN HAASE, HAMBURG (DE); ARJEN VAN DER HORST, TILBURG (NL); HANNES NICKISCH, HAMBURG (DE); HOLGER SCHMITT, LUETJENSEE (DE); MANINDRANATH VEMBAR, TWINSBURG, OH (US)

(21) Appl. No.: 19/116,682

(22) PCT Filed: Sep. 21, 2023
(86) PCT No.: PCT/EP2023/075999

§ 371 Date: Mar. 28, 2025

Related U.S. Application Data
(60) Provisional application No. 63/412,557, filed on Oct. 3, 2022.

(51) Int. Cl. A61B 34/10

(52) U.S. Cl. CPC A61B 34/10; A61B 2034/104; A61B 2034/105; A61B 2034/107

(57) ABSTRACT

A method and system are provided for planning a medical intervention, such as a coronary intervention. At least one image is retrieved, where the image includes at least a portion of a coronary artery. Based on the at least one image, a position and composition of plaque in the coronary artery are determined. A mechanical model of the portion of the coronary artery and the plaque in the coronary artery is generated, and a plurality of potential interventions is simulated in the context of the mechanical model. Following such simulations, an intervention for implementation is selected from the plurality of potential interventions.

### FIELD OF THE INVENTION

The present disclosure generally relates to systems and methods for planning coronary interventions, such as coronary stenting, in order to improve quality, speed, and outcomes for such procedures. In particular, the present disclosure relates to the use of computed tomography (CT) plaque characterization generated through non-invasive coronary CT angiography in procedure simulations in order to plan interventions.

### BACKGROUND

Coronary interventions, such as stenting, are often guided by intraprocedural imaging. Such imaging may be via invasive coronary angiography with X-ray and contrast agent or, in the case of more complex procedures, such angiography may be complemented by intravascular measurements, such as pressure or flow measurements, or intravascular imaging, such as intravascular ultrasound (IVUS) or optical coherence tomography (OCT).

Plaque location and composition may be utilized to estimate risk associated with a particular intervention, as well as to plan an intervention in terms of appropriate device type, size, or sequence. Similarly, information on coronary plaque may be used to decide if a specific coronary procedure is likely to be successful at all or if a different treatment strategy should be chosen, such as coronary artery bypass graft surgery (CABG). However, only limited information on plaque location or composition are available during the intervention. Accordingly, plaque location and composition are typically not used for planning or guiding such interventions today.

As such, there is a need for a system and pre-operative method for utilizing plaque analysis for determining an appropriate coronary intervention, as well as for planning a particular intervention. There is a further need for such a system and method that can continue to guide an intervention once selected.

### SUMMARY

Methods and systems are provided for planning coronary interventions, such as coronary stenting, in order to improve quality, speed, and outcomes for such procedures. Embodiments described herein use CT plaque characterization generated through imaging, such as non-invasive coronary CT angiography in procedure simulations in order to plan interventions.

Non-invasive imaging using computed tomography can be used to derive information about the plaque location, type, and composition, as well as derive physical parameters for a coronary segment or the coronary artery tree. This information is then utilized with respect to treatment guidance and device selection. In many embodiments, such device selection relates specifically to stenting coronary arteries. The systems and methods described herein can help decide upon stent length, diameter, positioning, and type of stent when treating a vessel segment.

Accordingly, the systems and methods described herein use imaging based plaque analysis prior to performing an intervention in order to determine the amount, type, shape, and/or location of plaque in the segment. Such imaging may be CT based, and the analysis may include Hounsfield unit (HU) imaging, mono-energetic images (MonoE), Zeff imaging, Calcium imaging, and iodine content imaging.

Such imaging may then be utilized for plaque analysis, and the analysis may be used to optimize the device selection and treatment parameters for the intervention.

In some embodiments, a method is provided for planning a medical intervention, such as a coronary intervention, the method including first retrieving at least one image, where the image includes at least a portion of a coronary artery. The method proceeds to determine, based on the at least one image, a position and composition of plaque in the coronary artery.

The method then proceeds to generate a mechanical model of the portion of the coronary artery and the plaque in the coronary artery, and simulates a plurality of potential interventions in the context of the mechanical model. Following such simulations, the method selects an intervention for implementation from the plurality of potential interventions.

In some embodiments, the plurality of simulated potential interventions includes a first intervention in which a first device is implanted and a second intervention in which a second device different from the first device is implanted. For example, the first device and the second device may each be stents selected from a group of bare metal stents, drug eluting stents, polymer stents, and bioresorbable stents. The first device and the second device may each have a different length or a different diameter.

In some embodiments, the plurality of simulated potential interventions may include a first intervention in which a stent is implanted and a second intervention comprising a coronary artery bypass graft surgery (CABG).

In some embodiments, the method includes identifying, in the image, a plurality of lesions to be addressed in the coronary intervention. The plurality of simulated potential interventions then includes a first intervention comprising a first sequence of lesion treatment and a second intervention comprising a second sequence of lesion treatment different from the first.

In some embodiments, the plurality of simulated potential interventions includes a first intervention in which a first balloon pressure is used to place a stent and second intervention in which a second balloon pressure different from the first balloon pressure is used to place the stent.

In some embodiments, the method further includes the implementation of the selected intervention. During such an implementation, the method may further include displaying the mechanical model on a display during implementation of the selected intervention.

In some such embodiments, the method may further include determining that the position or composition of the plaque has changed during implementation of the selected intervention. The method may then execute a real time evaluation of potential complications based on an updated position or composition of the plaque.

In some embodiments in which the model is displayed during performance of the intervention, the intervention utilizes a device, and the method includes monitoring a position of the device and displaying a risk metric on the display during implementation of the selected intervention. The risk metric may be at least partially based on a precise location of the device during the selected intervention.

In some embodiments, the determination of the position and composition of the plaque is based on an artificial intelligence (AI) model. In some such embodiments, the composition of the plaque defines a stiffness or deformability of the plaque. The mechanical model of the portion of the coronary artery and the plaque in the coronary artery simulates deformation of the portion of the coronary artery in response to mechanical force based on the composition of the plaque.

In some such embodiments, the mechanical model may further simulate stress and strain as isometric forces resulting from the use of a stent or balloon.

In some embodiments, the simulation of the plurality of potential interventions is based on an AI model trained based on results of previous intervention implementations.

In some embodiments, the determining of the composition of the plaque in the coronary artery includes quantifying calcium content, density, or fibrous components. In some such embodiments, the method further includes classifying the plaque as positive modeling plaque, lumen narrowing plaque, or a thrombus.

Also provided is a system for performing a coronary intervention. Such a system includes a plurality of potential devices available for implantation, a memory for storing a plurality of instructions, and a processor circuitry that couples with the memory.

The processor circuitry is configured to execute instructions to first retrieve at least one image, the image including at least a portion of the coronary artery and determine, based on the image, a position and composition of plaque in the coronary artery.

The processor then proceeds to generate a mechanical model of the portion of the coronary artery and the plaque in the coronary artery, and simulates a plurality of potential interventions in the context of the mechanical model. Each of the potential interventions utilizes at least one of the plurality of potential devices.

The processor then proceeds to select an intervention for implementation from the plurality of potential interventions.

In some embodiments, the plurality of potential devices are stents, each selected from a group of bare metal stents, drug eluting stents, polymer stents, and bioresorbable stents. At least two of the plurality of potential devices have a different length or diameter from each other.

In some embodiments, the plurality of simulated potential interventions includes a first intervention in which a first balloon pressure is used to place a stent and a second intervention in which a second balloon pressure different from the first balloon pressure is used to place the stent.

In some embodiments, the determination of the position and composition of the plaque is based on an AI model. In some such embodiments, the composition of the plaque defines a stiffness or deformability of the plaque. The mechanical model of the portion of the coronary artery and the plaque in the coronary artery then simulates deformation of the portion of the coronary artery in response to mechanical force based on the composition of the plaque.

### BRIEF DESCRIPTION OF THE DRAWINGS

FIG. 1 is a schematic diagram of a system according to one embodiment of the present disclosure.

FIG. 2 illustrates a method for processing images in accordance with this disclosure.

FIG. 3 illustrates the use of a plaque model during an intervention in accordance with the method of FIG. 2.

### DETAILED DESCRIPTION OF THE PREFERRED EMBODIMENTS

The description of illustrative embodiments according to principles of the present invention is intended to be read in connection with the accompanying drawings, which are to be considered part of the entire written description. In the description of embodiments of the invention disclosed herein, any reference to direction or orientation is merely intended for convenience of description and is not intended in any way to limit the scope of the present invention. Relative terms such as “lower,” “upper,” “horizontal,” “vertical,” “above,” “below,” “up,” “down,” “top” and “bottom” as well as derivative thereof (e.g., “horizontally,” “downwardly,” “upwardly,” etc.) should be construed to refer to the orientation as then described or as shown in the drawing under discussion. These relative terms are for convenience of description only and do not require that the apparatus be constructed or operated in a particular orientation unless explicitly indicated as such. Terms such as “attached,” “affixed,” “connected,” “coupled,” “interconnected,” and similar refer to a relationship wherein structures are secured or attached to one another either directly or indirectly through intervening structures, as well as both movable or rigid attachments or relationships, unless expressly described otherwise. Moreover, the features and benefits of the invention are illustrated by reference to the exemplified embodiments. Accordingly, the invention expressly should not be limited to such exemplary embodiments illustrating some possible non-limiting combination of features that may exist alone or in other combinations of features; the scope of the invention being defined by the claims appended hereto.

This disclosure describes the best mode or modes of practicing the invention as presently contemplated. This description is not intended to be understood in a limiting sense, but provides an example of the invention presented solely for illustrative purposes by reference to the accompanying drawings to advise one of ordinary skill in the art of the advantages and construction of the invention. In the various views of the drawings, like reference characters designate like or similar parts.

It is important to note that the embodiments disclosed are only examples of the many advantageous uses of the innovative teachings herein. In general, statements made in the specification of the present application do not necessarily limit any of the various claimed disclosures. Moreover, some statements may apply to some inventive features but not to others. In general, unless otherwise indicated, singular elements may be in plural and vice versa with no loss of generality.

A pre-operative method is described for improving the quality, speed, and outcome of coronary interventions based on plaque characterizations prior to such interventions. The plaque characterization is based on images, such as coronary CT angiography, performed prior to the procedure.

Typically, if a patient is scheduled for a coronary intervention, the method described herein may be applied to identify an ideal form of that intervention. This may include choosing a type of intervention from several available types, and it may include choosing specific implantable devices for use in the intervention. For example, the method described herein may be used to determine which of several available stents should be implanted, as well as to determine an ideal balloon pressure to be used during implantation.

Accordingly, once a category of intervention is determined to be appropriate, the method involves selecting an appropriate implant, and in some embodiments, guiding the actual implanting of an appropriate device during the intervention.

The method involves generating a mechanical model of the portion of the coronary artery that requires intervention, along with plaque residing therein. The method then further involves simulating potential interventions in the context of that mechanical model in order to evaluate potential risks associated with multiple potential interventions.

As such, the method typically involves retrieving an image of at least a portion of the coronary artery requiring intervention. Such an image is typically non-invasive coronary CT angiography, and may include spectral CT imaging. In many embodiments, such CT angiography is performed prior to the intervention and is therefore retrieved by a processor implementing the method, while in other embodiments, a system implementing the method includes an imaging system for generating the imaging. The CT angiography is then analyzed to evaluate plaque content, and such analysis may include Hounsfield unit (HU) imaging, mono-energetic images (MonoE), Zeff imaging, Calcium imaging, and iodine content imaging.

The plaque content may be used to generate the mechanical model used to plan and evaluate the intervention. As such, characteristics of the coronary artery, such as stiffness, may be informed by the plaque location and composition.

Further, in some embodiment, pre-intervention imaging may take a form other than CT. In medical imaging other than CT, such as Magnetic Resonance Imaging (MRI) or Positron Emission Tomography (PET), different methods may be used for processing images, and resulting images may take different forms. In this disclosure, embodiments are discussed in terms of CT imaging. However, it will be understood that the methods and systems described herein may be used in the context of other imaging modalities as well.

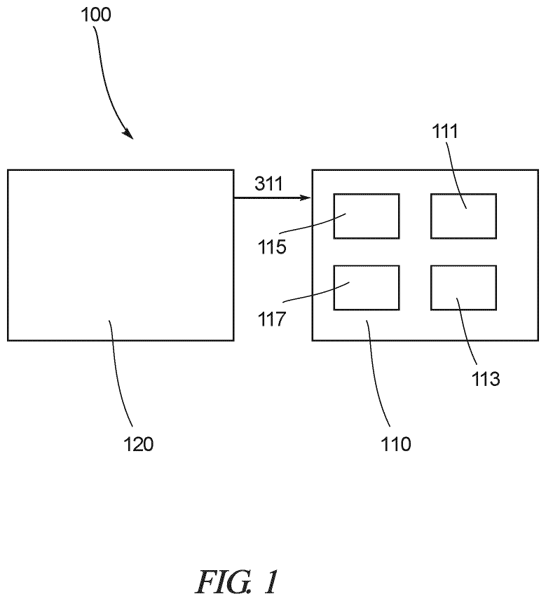

FIG. 1 is a schematic diagram of a system 100 according to one embodiment of the present disclosure. As shown, the system 100 typically includes a processing device 110 and an imaging device 120.

The processing device 110 may apply processing routines to images or measured data, such as projection data, received from the imaging device 120. The processing device 110 may include a memory 113 and processor circuitry 111. The memory 113 may store a plurality of instructions. The processor circuitry 111 may couple to the memory 113 and may be configured to execute the instructions. The instructions stored in the memory 113 may comprise processing routines, as well as data associated with processing routines, such as machine learning algorithms, and various filters for processing images. While all data is described as being stored in the memory 113, it will be understood that in some embodiments, some data may be stored in a database, which may itself either be stored in the memory or stored remotely (for example, cloud-based) in a discrete system.

The processing device 110 may further include an input 115 and an output 117. The input 115 may receive information, such as images or measured data, from the imaging device 120. The output 117 may output information, such as processed images, to a user or a user interface device. The output 117 similarly may output determinations generated by the method described below, such as recommendations and risk determinations. The output may include a monitor or display which may display additional information or a model updated in real-time.

In some embodiments, the processing device 110 may relate to the imaging device 120 directly. In alternate embodiments, the processing device 110 may be distinct from the imaging device 120, such that it receives images or measured data for processing by way of a network or other interface at the input 115.

In some embodiments, the imaging device 120 may include an image data processing device, and a spectral, photon counting, or conventional CT scanning unit for generating the CT projection data when scanning an object (e.g., a patient). Further, the imaging device 120 may be set up for either invasive or non-invasive coronary CT angiography. As such, the imaging may be performed with contrast, and the image timing may be set up in order to track fluid flow in blood vessels.

In addition to conventional and spectral CT images, the method may rely on multiple spectral image results, photon counting CT images, or dark field CT images.

While a system is shown including an imaging device 120 and a processing device 110, it will be understood that the method may be implemented directly on a processing device, as in the context of an image received by way of a network at the input 115. The methods described herein involve processing an image as a component of evaluating and planning potential interventions, generally in the context of a procedure, such as stenting. As noted above, typically, prior to such a procedure, imaging is performed. As such, previously generated imaging may be retrieved by way of the input 115 and evaluated prior to or in place of obtaining a new image.

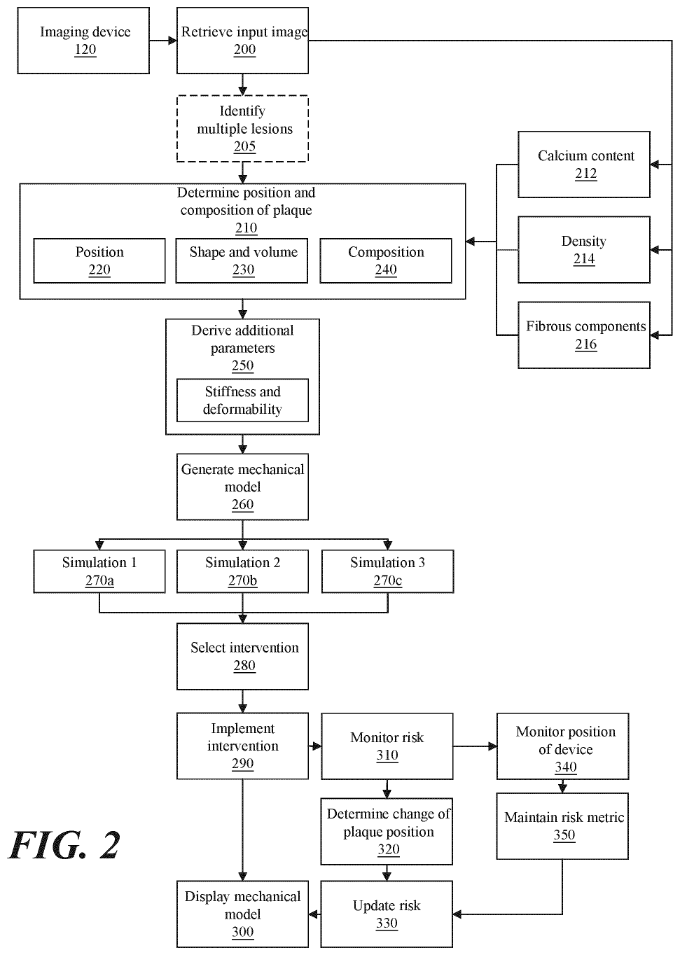

FIG. 2 illustrates a method for processing images in accordance with this disclosure. As shown, in implementing the method, the system 100 may first retrieve (200), at an input 115, at least one image. The image includes at least a portion of a coronary artery of the patient. The retrieved image is typically CT imaging, such as non-invasive coronary CT angiography, which may be used to evaluate plaque in the coronary artery.

In some embodiments, prior to evaluating plaque content of the coronary artery, the method may identify (205) one or more lesion in the image, where the identified lesions require potential intervention.

The method may then determine (210), based on the at least one image, a position and composition of plaque in the coronary artery. The plaque may be defined in terms of location, distribution, and composition. Such determination may be by manual or automatic detection, classification, and segmentation. Accordingly, the determination of the position and composition of the plaque may be based on an AI model, such as a convolutional neural network (CNN).

In embodiments in which multiple lesions have been identified (at 205), the plaque may then be located, as part of the determination (210) relative to each lesion, or relative to the coronary artery taken as a whole.

Accordingly, the determination (at 210) may include defining the position 220 of the plaque along the coronary artery, the shape and volume of the plaque 230 at that position, and the composition of the plaque 240. The composition may be determined based on various strategies, such as using Hounsfield unit (HU) imaging, mono-energetic images (MonoE), Zeff imaging, Calcium imaging, or iodine content imaging. This may be used to classify the composition of the plaque using a mean, a histogram, or by dividing the plaque into sub-volumes of different plaque components.

The determination may also classify the type of plaque by identification of, for example, positive remodeling plaque, lumen narrowing plaque, or a thrombus.

In order to support such determinations, different types of plaque may be evaluated based on the images, and the determination of the composition of plaque (at 210) may include quantifying calcium content 212, density 214, or fibrous components 216.

In some embodiments, calcium content quantification may be in terms of CAN=Non-Calcified/CAL=Low or mixed level of Calcification/CAH=Highly Calcified.

High-Risk Plaque (HRP) features may be identified as well. Such high risk features may include LA=Low Attenuation plaque <30HU/NR=Napkin-Ring-sign i.e. (low attenuation core surrounded by rim-like higher attenuation and <130HU)/PR=Positive Remodeling (remodeling idx >1.1=10% increase in CSA)/SC=Spotty Calcification (calcified foci between 1-3 mm and >130HU) Other patterns may be identifiable as well. For example, plaque may be classified as DC=Dense Calcification (massively calcified, other plaque components negligible)/FM=Fibro-calcified mix (mixture of calcified foci and fibrous components)/FP=Fibrous Plaque (exclusively fibrous, other plaque components negligible) Other features of interest may include Thrombus/Peri-coronary inflammation (e.g., via pFAI).

Additional parameters may then be derived (250) from the plaque data, such as the distribution of plaque along the vessel centerline, the angular coverage of the plaque in a cross section of the coronary artery, a portion of the lumen occupied by the plaque when viewed in cross-section, the distance to the next bifurcation of the vessel, and the stiffness or deformability of the plaque.

The method then proceeds to generate (260) a mechanical model of the portion of the coronary artery and the plaque in the coronary artery. Such a mechanical model may then rely on the characteristics or classification of the plaque generated as part of the determination (at 210), as well as the additional parameters (derived at 250) in order to accurately model responses to various interventions. The mechanical model may be a numerical model (lumped element or finite element model or finite volume or finite difference or meshfree numerical method or couple computation fluid dynamics model) of the plaque and vessel segment.

For example, additional parameters (at 250) may rely on the composition of the plaque to define stiffness or deformability of the plaque. The plaque data may then be combined with anatomic parameters describing the vessel segment. For example, an AHA model of the blood vessel may be relied upon, if available. The mechanical model may then consider the position of the plaque along the centerline, the distance to the coronary ostium and/or next bifurcation, local curvature at the plaque position, as well as curvature in the neighborhood (i.e., tortuosity).

The mechanical model may then simulate deformation of the coronary artery in response to mechanical force based on the composition of the plaque. For example, the mechanical model may be used to simulate mechanical forces resulting from the use of a stent or balloon. The balloon may then be simulated with different pressure levels.

Once the mechanical model is generated (at 260), the method proceeds with simulating (270a, 270b, 270c) a plurality of potential interventions in the context of the mechanical model. Such simulations may be performed based on an AI model, such as a CNN. Such an AI model may be trained on training data, which may include results of previous intervention implementations in the context of similar plaque determinations.

Once such interventions are simulated, the method selects (280) one such intervention for implementation.

The simulated potential interventions may include multiple distinct types of interventions or interventions including distinct devices for implantation. Alternatively, the potential interventions may be similar procedures utilizing different devices. For example, the potential interventions may include implantations of stents of different sizes or types, or implantations utilizing different balloon pressures.

For example, in some embodiments, a first intervention 270a of the plurality of potential interventions may be implantation of a first device and a second intervention 270b may be implantation of a second device different from the first device.

The first and second devices may then each be stents selected from a group of bare metal stents, drug eluting stents, polymer stents, and bioresorbable stents. Further, the first and second devices may have different lengths or diameters. For example, a length may be selected to remove the stenosis and to cover neighboring plaque.

Similarly, a first intervention 270a may be implantation of a stent in which a first balloon pressure is used to place the stent, and a second intervention 270b relies on a second balloon pressure different from the first balloon pressure in order to place the stent.

Alternatively, in some embodiments a first intervention 270a may be an implantation of a stent, and a second intervention 270b may be a coronary artery bypass graft surgery.

In embodiments in which multiple lesions have been identified (at 205), and in which a plurality of lesions are to be addressed in the coronary intervention being evaluated, the plurality of potential interventions 270a, 270b, 270c to be simulated may include a first intervention 270a comprising a first sequence of lesion treatment and a second intervention 270b comprising a second sequence of lesion treatment different from the first sequence.

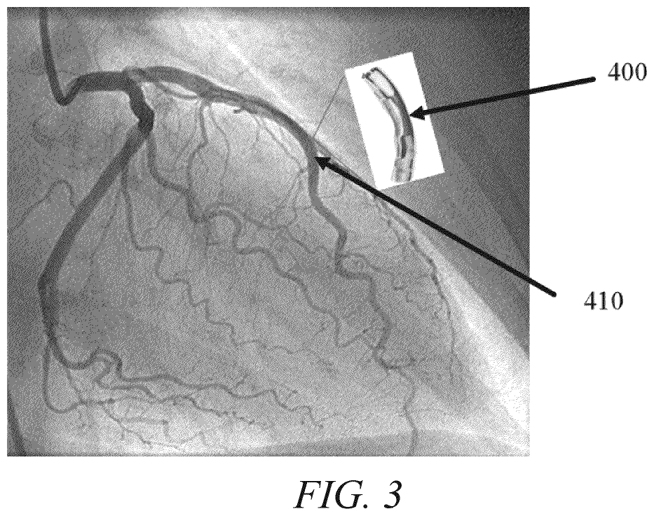

FIG. 3 illustrates the use of a plaque model 400 during an intervention in accordance with the method of FIG. 2. In some embodiments, following the selection of an intervention for implementation (at 280), the method proceeds with actual implementation (290) the intervention selected 270a. In such embodiments, the method may then display (300) the mechanical model on a display during the implementation of the intervention 270a. Accordingly, as shown in FIG. 3, the mechanical model 400 may be shown in the context of the coronary artery 410 during the intervention.

For example, the mechanical model 400 may be an overlay to coronary angiography images. The display may include different levels of information, such as image derived parameters of the plaque or results of models generated based on the image derived plaque parameters. The information may similarly be displayed on top of intravascular images. Additionally, the information derived may be presented to a user as a type of worklist.

The method may continue to monitor (310) risk associated with the intervention during implementation, and may alert a user to any change in the risk model. For example, in some embodiments, the method may monitor continued imaging or may otherwise track plaque within the coronary artery. The method may then determine (320) that the position or composition of the plaque has changed during implementation of the selected intervention. The method may then proceed to execute a real time evaluation (330) of potential complications based on an updated position or composition of the plaque. In such embodiments, the method may continue to guide the intervention based on the mechanical model updated to reflect any changes, as well as alerting the user to any new or modified risk associated with the intervention.

In some embodiments, the plaque information or plaque biomarkers derived from the plaque data can be used to predict risk of a particular intervention. Accordingly, the method may make recommendations with respect to steps involved in a particular intervention, or a user may provide an indication as to what steps are to be performed on a particular patient. The method may then provide an indication of risk level.

In some embodiments, where the selected intervention utilizes a device, the precise location of the device during the intervention may impact risk. As such, the monitoring performed by the method (at 310) may be paired with monitoring (340) a position of the device during the intervention. The method may then track and/or display a risk metric (350), where the risk metric is at least partially based on the precise position of the device during the selected intervention 270a.

Similarly, the risk level may be displayed when a device, a guidewire, or a similar locating element, reaches a certain position in the vascular tree, from which it can be concluded where the next steps of the procedure will be carried out. The method may then update a risk model accordingly.

Risk prediction in this context may include risk of procedure complication or risk of a cardiac event (i.e., MACE) in the next 1, 2, or 5 years after the intervention. Risk prediction may further consider quality of life after intervention.

The joint system described herein, namely CT imaging combined with plaque characterization and an interventional procedure, can be used to train an AI based system predicting the procedure steps and parameters to treat a specific lesion in future implementations. After plaque characterization, the device used to treat the lesion with the plaque or other parameters like the balloon pressure may then be stored in a database. Based on such data, the system may then be trained to predict, given a specific plaque configuration in a vessel, which devices shall be used and what risk is associated with the procedure.

A system is similarly provided for implementing the method described. Such a system may include a plurality of potential devices made available for implementation, depending on an intervention to be utilized. Further, as shown in FIG. 1, such a system may include a memory 113 for storing a plurality of instructions and processor circuitry 111 that couples with the memory and is configured to execute instructions for performing the method described above with respect to FIG. 2.

When performing the method, it is noted that upon simulating the plurality of potential interventions 270a, 270b, 270c in the context of the mechanical model (generated at 260), each of the potential interventions utilizes at least one of the plurality of potential devices made available for implementation.

While the system and method are described herein in terms of coronary arteries, a similar method may applied to other vascular structures, such as peripheral or pulmonary vessels, carotid arteries, and others. Similarly, as discussed above, a system implementing the method may include various forms of computing. As such, the processing may be carried out on premises or on a cloud computing platform. Predictions and intervention planning may be displayed on the CT console, in an advanced workstation, or in the interventional lab prior to or during the procedure selected.

The method may be used in CT systems and imaging workstations and PACS viewers dedicated to coronary analysis using CCTA scans, as well as for interventional C-arm systems when the information is used intraprocedural.

In some cases, the imaging retrieved (at 200) may be insufficient for the type of planning required. For example, non-invasive imaging retrieved may be low resolution or otherwise sub-optimal, but may be sufficient for providing an indication that there is a plaque-associated risk. The method may then use and guide further intravascular imaging prior to the treatment.

Similarly, if additional information is available, such information may be incorporated into the various analyses discussed herein. For example, if non-invasive imaging of more than one cardiac phase is available, thereby providing temporal information, such information on the plaque dynamics may be included in the analysis.

The methods according to the present disclosure may be implemented on a computer as a computer implemented method, or in dedicated hardware, or in a combination of both. Executable code for a method according to the present disclosure may be stored on a computer program product. Examples of computer program products include memory devices, optical storage devices, integrated circuits, servers, online software, etc. Preferably, the computer program product may include non-transitory program code stored on a computer readable medium for performing a method according to the present disclosure when said program product is executed on a computer. In an embodiment, the computer program may include computer program code adapted to perform all the steps of a method according to the present disclosure when the computer program is run on a computer. The computer program may be embodied on a computer readable medium.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

All examples and conditional language recited herein are intended for pedagogical purposes to aid the reader in understanding the principles of the disclosure and the concepts contributed by the inventor to furthering the art, and are to be construed as being without limitation to such specifically recited examples and conditions. Moreover, all statements herein reciting principles, aspects, and embodiments of the disclosure, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

1. A method for planning a coronary intervention, comprising:
retrieving at least one image, the at least one image including at least a portion of a coronary artery;
determining, based on the at least one image, a position and composition of plaque in the coronary artery;
generating a mechanical model of the portion of the coronary artery and the plaque in the coronary artery;
simulating a plurality of potential interventions in the context of the mechanical model; and
selecting an intervention for implementation from the plurality of potential interventions.

2. The method of claim 1, wherein the plurality of simulated potential interventions includes a first intervention in which a first device is implanted and a second intervention in which a second device different from the first device is implanted.

3. The method of claim 2, wherein the first device and the second device are stents, each selected from a group of bare metal stents, drug eluting stents, polymer stents, and bioresorbable stents, and wherein the first device and the second device each have a different length or diameter.

4. The method of claim 1, wherein the plurality of simulated potential interventions includes a first intervention in which a stent is implanted and a second intervention comprising a coronary artery bypass graft surgery (CABG).

5. The method of claim 1, further comprising identifying, in the image, a plurality of lesions to be addressed in the coronary intervention, and wherein the plurality of simulated potential interventions includes a first intervention comprising a first sequence of lesion treatment and a second intervention comprising a second sequence of lesion treatment different from the first sequence.

6. The method of claim 1, wherein the plurality of simulated potential interventions includes a first intervention in which a first balloon pressure is used to place a stent and a second intervention in which a second balloon pressure different from the first balloon pressure is used to place the stent.

7. The method of claim 1, further comprising implementing the selected intervention and displaying the mechanical model on a display during implementation of the selected intervention.

8. The method of claim 7, further comprising determining whether the position or composition of the plaque has changed during implementation of the selected intervention and executing a real time evaluation of potential complications based on an updated position or composition of the plaque.

9. The method of claim 7, wherein the selected intervention utilizes a device, the method further comprising monitoring a position of the device and displaying a risk metric on the display during implementation of the selected intervention, wherein the risk metric is at least partially based on a precise position of the device during the selected intervention.

10. The method of claim 1, wherein the determination of the position and composition of the plaque is based on an AI model.

11. The method of claim 10, wherein the composition of the plaque defines a stiffness or deformability of the plaque, and wherein the mechanical model of the portion of the coronary artery and the plaque in the coronary artery simulates deformation of the portion of the coronary artery in response to mechanical force based on the composition of the plaque.

12. The method of claim 11, wherein the mechanical model simulates stress and strain as isometric forces resulting from the use of a stent or balloon.

13. The method of claim 1, wherein the simulation of the plurality of potential interventions is based on an AI model trained based on results of previous intervention implementations.

14. The method of claim 1, wherein the determining of the composition of plaque in the coronary artery includes quantifying calcium content, density, or fibrous components.

15. The method of claim 14, further comprising classifying the plaque as positive remodeling plaque, lumen narrowing plaque, or a thrombus.

16. A system for performing a coronary intervention comprising:
a memory for storing a plurality of instructions;
processor circuitry that couples with the memory and is configured to execute the instructions to:
retrieve at least one image, the at least one image including at least a portion of the coronary artery;
determine, based on the at least one image, a position and composition of plaque in the coronary artery;
generate a mechanical model of the portion of the coronary artery and the plaque in the coronary artery;
simulate a plurality of potential interventions in the context of the mechanical model, each of the potential interventions utilizing at least one of the plurality of potential devices; and
select an intervention for implementation from the plurality of potential interventions.

17. The system of claim 16, further comprising a plurality of potential devices for implantation during the selected intervention, wherein the plurality of potential devices are stents, each selected from a group of bare metal stents, drug eluting stents, polymer stents, and bioresorbable stents, and wherein at least two of the plurality of potential devices have a different length or diameter from each other.

18. The system of claim 16, wherein the plurality of simulated potential interventions includes a first intervention in which a first balloon pressure is used to place a stent and a second intervention in which a second balloon pressure different from the first balloon pressure is used to place the stent.

19. The system of claim 16, wherein the determination of the position and composition of the plaque is based on an AI model.

20. The system of claim 19, wherein the composition of the plaque defines a stiffness or deformability of the plaque, and wherein the mechanical model of the portion of the coronary artery and the plaque in the coronary artery simulates deformation of the portion of the coronary artery in response to mechanical force based on the composition of the plaque.
